(19) United States
(12) Patent Application Publication
(10) Pub. No.: US 2010/0036210 A1
(43) Pub. Date: Feb. 11, 2010

(54) ELASTIC CLOTH DEVICE WITH A FUNCTION OF PHYSIOLOGICAL DETECTION

(75) Inventor: TONG-PIE CHEN, SANCHUNG CITY (TW)

Correspondence Address: KILE GOEKJIAN REED & MCMANUS, 1200 NEW HAMPSHIRE AVE, NW, SUITE 570, WASHINGTON, DC, 20036, US

(73) Assignee: ZENTAN TECHNOLOGY CO., LTD.

(21) Appl. No.: 12/411,653

(22) Filed: Mar. 26, 2009

(30) Foreign Application Priority Data
Aug. 6, 2008 (TW) 97214062

(51) Int. Cl. A61B 5/00

(52) U.S. Cl. 600/301

(57) ABSTRACT

An elastic cloth device with a function of physiological detection includes two elastic bodies, two fastening elements, and a connecting strap. The two elastic bodies are aligned with an interval in-between and each includes an elastic cloth strap woven with at least one strain sensing unit; wherein the strain sensing unit has a surface emerging from the elastic cloth strap. Each fastening element is combined with one end of the corresponding elastic body and electrically coupled with the corresponding strain sensing unit. The connecting strap is connected with the ends of the elastic bodies so that the elastic bodies are spacedly connected in series. Accordingly, when a physiological strain reaction has an effect on the elastic cloth straps, the straps drive the strain sensing units to produce a physiological strain signal which is electrically transmitted to the fastening elements, thereby the function of physiological detection is achieved.

### CROSS-REFERENCE TO RELATED APPLICATION

This application claims priority under 35 U.S.C. §119 to Taiwan Patent Application No. 097214062, filed on Aug. 6, 2008, in the Taiwan Intellectual Property Office, the entire contents of which are hereby incorporated by reference.

### BACKGROUND OF THE INVENTION

1. Field of the Invention

The present invention relates to a sensing device, in particular to an elastic cloth device with a function of physiological detection for detecting physiological signals.

2. Description of Related Art

With the improvement of people's living standards, people pay more and more attention to their health. People usually use all kinds of physiological measurement apparatus to detect their current physiological parameters at all times so as to remain informed and thereby adjust their health conditions properly.

However, physiological detection has many detection types, such as ECG (electrocardiogram), body temperatures, brain waves, pulses, and so on. A conventional physiological detection device, for example, a glove-typed physiological measurement device disclosed in Taiwan Patent Certificate No. M301046 (Application No. 95208968), includes a glove body, a physiological measurer, and a physiological detector. The glove body is worn on a user's hand, and electrical connection bases are respectively disposed on the back of the hand and the fingers. Electric circuitries electrically couple between the electrical connection bases respectively. The physiological measurer has electrical connectors engaging with the electrical connecting bases on the back of the hand and the physiological detector also has electrical connectors engaging with the electrical connecting bases on the fingers, so that the physiological detector can contact the user's fingers and obtain pulse signals, and then transmit the pulse signals to the physiological measurer to show the measured value to the user.

However, the glove-typed physiological measurement device still has some shortcomings. For example, except for hands, it cannot be used on other body parts such as wrists, chests, and so on, so the glove-typed physiological measurement device has a narrow usage range. Furthermore, the physiological detector has a small contact area with skins, so that the obtained signals according to the physiological strain reactions always have limited strength and the noise is too high, therefore accurate physiological signals are difficult to obtain

Hence, the inventors of the present invention believe that the shortcomings described above are able to be improved and finally suggest the present invention which is of a reasonable design and is an effective improvement based on extensive research and thought.

### SUMMARY OF THE INVENTION

An object of the present invention is to provide an elastic cloth device with a function of physiological detection which can provide elasticity to be worn on a user's body during use, thereby achieving a better usage effect and comfortableness and adaptable to more parts of the body for physiological detection. The elastic cloth device further has large contact areas so that detection signals according to physiological strain reaction of the user can have greater strength so as to detect physiological signals more accurately.

To achieve the above-mentioned object, an elastic cloth device with a function of physiological detection in accordance with the present invention is provided. The elastic cloth device includes an elastic body having an elastic cloth strap and at least one strain sensing unit elastically woven with the elastic cloth strap, the strain sensing unit having a surface emerging from the elastic cloth strap; and a fastening element with electrical conductivity combined on the elastic body and electrically coupled with the strain sensing unit; whereby when a physiological strain reaction has an effect on the elastic cloth strap, the elastic cloth strap drives the strain sensing unit to produce a physiological strain signal and the physiological strain signal is electrically transmitted to the fastening element.

To achieve the above-mentioned object, an elastic cloth device with a function of physiological detection in accordance with the present invention is further provided. The elastic cloth device with a function of physiological detection includes two elastic bodies adjacent to each other at intervals in turn and each including an elastic cloth strap and at least one strain sensing unit woven with the elastic cloth strap, the strain sensing unit having a surface emerging from the elastic cloth strap; two fastening elements with electrical conductivity each combined with one end of the corresponding elastic body and electrically coupled with the corresponding strain sensing unit; and a connecting strap connected with the ends of the elastic bodies so that the elastic bodies are spacedly connected in series; whereby when a physiological strain reaction has an effect on the elastic cloth straps, the elastic cloth straps drive the strain sensing units to produce a physiological strain signal and the physiological strain signal is electrically transmitted to the fastening elements.

The efficacy of the present invention is as follows: based on the elastic tension of the elastic bodies, the elastic cloth device of the present invention can be securely attached to users' bodies according to users' body types, thereby achieving a better usage effect and being adapted for more desired detected parts. Further, the strain sensing units also have elasticity, so they can elastically cling to users' skins to detect the correct physiological signals; and the strain sensing units also have a larger contact areas so that detection signals according to the user's physiological strain reaction can have greater strength so as to detect users' physiological signals more accurately and steadily.

To further understand features and technical contents of the present invention, please refer to the following detailed description and drawings related the present invention. However, the drawings are only to be used as references and explanations, not to limit the present invention.

### BRIEF DESCRIPTION OF THE DRAWINGS

FIG. 1 is a plan view of an elastic cloth device with a function of physiological detection of the present invention;

FIG. 2 is a perspective view of the elastic cloth device with a function of physiological detection of the present invention;

FIG. 3 is a schematic view of the elastic cloth device with a function of physiological detection of the present invention in a using state;

FIG. 4 is a perspective view of an alternative embodiment of the elastic cloth device with a function of physiological detection of the present invention;

FIG. 5 is a schematic view of the alternative embodiment of the elastic cloth device with a function of physiological detection of the present invention in a using state; and

FIG. 6 is a perspective view of another alternative embodiment of the elastic cloth device with a function of physiological detection of the present invention.

### DETAILED DESCRIPTION OF THE PREFERRED EMBODIMENTS

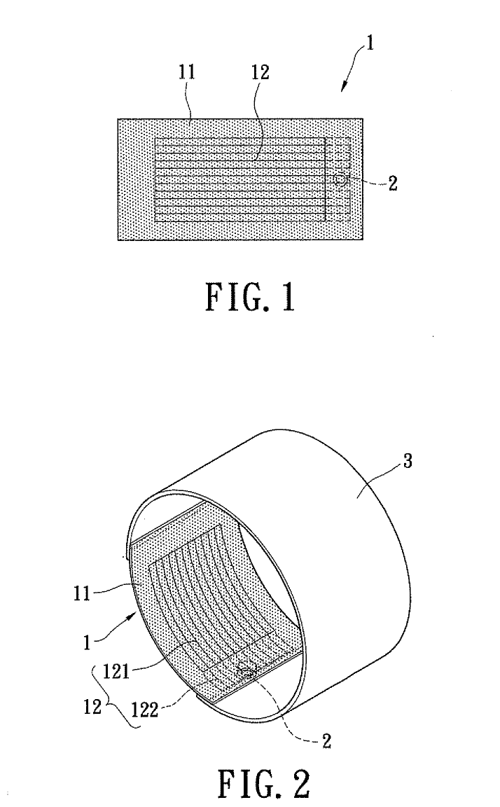

Please refer to FIG. 1 and FIG. 2, which illustrates an elastic cloth device with a function of physiological detection according to the present invention. The elastic cloth device includes at least one elastic body 1 and at least one fastening element 2.

The elastic body 1 includes an elastic cloth strap 11 and at least one strain sensing unit 12. The elastic cloth strap 11 can be elastic yarns, elastic cloth, or an elastic fabric woven by elastic yarns and elastic cloth. The strain sensing unit 12 is elastically woven with the elastic cloth strap 11, wherein the strain sensing unit 12 has a surface emerging from the elastic cloth strap 11.

In the present embodiment, the strain sensing unit 12 has a plurality of strain sensing conductive yarns and include a first portion 121 and a second portion 122 integrally and continuously connected with the first portion 121, wherein the second portion 122 is electrically conductive. The first portion 121 is woven with the elastic cloth strap 11, wherein the first portion 121 emerges from one surface of the elastic cloth strap 11. The second portion 122 is woven in the elastic cloth strap 11 and embedded in the elastic cloth strap 11, or in the elastic cloth strap 11 for a distance and then emerge from the surface of the elastic cloth strap 11. The present invention limited the above described, and the first portion 121 and the second portion 122 both emerge from the surface of the elastic cloth strap 11, that is, the second portion 122 directly extend to the fastening element 2 from the surface of the elastic cloth strap 11. On the other hand, the number of the strain sensing units 12 can be determined according to actual demands, for example, one strain sensing unit 12 is combined on the elastic cloth strap 11, or more than strain sensing units 12 are non-conductively combined on the elastic cloth strap 11 side by side.

The fastening element 2 is made of a conductive material. The fastening element 2 is a male fastening element or a female fastening element, or a plurality of male fastening elements or female fastening elements, wherein the fastening element 2 corresponds to the plurality of long-strip-shaped strain sensing units 12 that has been aforementioned. The fastening element 2 is combined on the elastic body 1 and electrically coupled with the strain sensing unit 12; concretely speaking, the electrical coupling is achieved via contacting the second portion 122 of the strain sensing unit 12 with the fastening element 2.

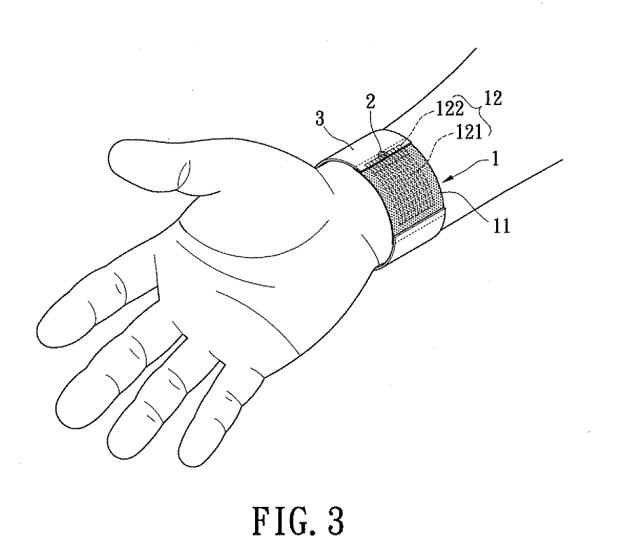

In an alternative embodiment of the present invention, please refer to FIG. 3, the elastic body 1 can be made into a ring body directly, or can be connected with a connecting strap 3 to form a ring body, thereby surrounding hands, wrists or joints etc. Accordingly, when a human body produces a physiological strain reaction which has an effect on the elastic cloth strap 11 of the elastic body 1, the elastic cloth strap 11 drives the strain sensing unit 12 to produce a physiological strain signal; wherein the physiological strain signal is transmitted to the fastening element 2 which can be fastened on a main control device (not shown) and electrically coupled with the main control device, so that the received physiological strain signal can be interpreted directly, or the main control device transmits the physiological strain signal to a display device by radio to detect physiological signals such as pulses, electrocardiograms, and so on.

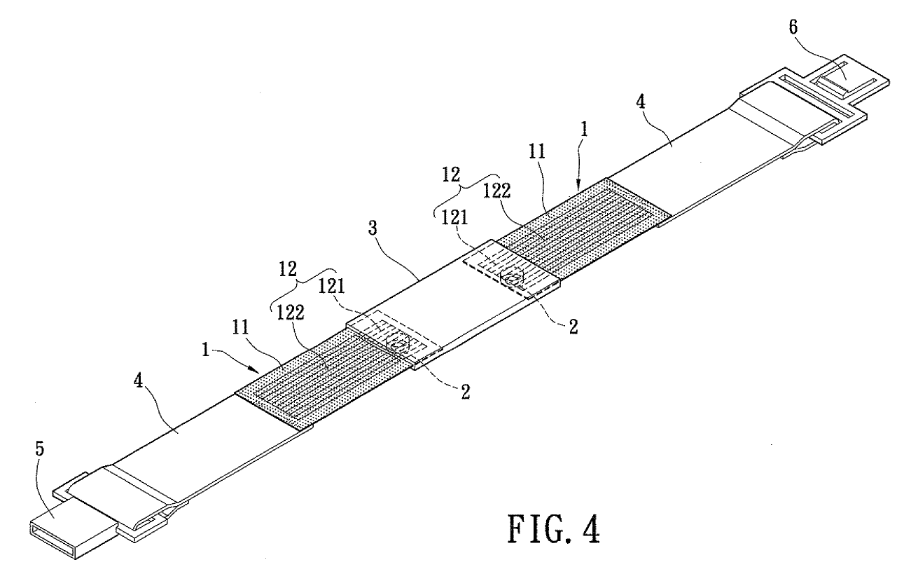

As shown in FIG. 4, in another alternative embodiment, the present invention further includes two elastic bodies 1, two fastening elements 2, and a connecting strap 3. The two elastic bodies are arranged at intervals in turn. The connecting strap 3 is connected to the ends of the two elastic bodies 1 so that the elastic bodies 1 are spacedly connected in series. Similarly, each of the two fastening elements 2 are combined with one end of the corresponding elastic body 1 and electrically coupled with the corresponding strain sensing unit 12. The fastening elements 2 are combined on the connecting portions where the connecting strap 3 and the elastic bodies 1 are connected. The strain sensing units 12 also have the features above described, so the detailed description is omitted.

Furthermore, each of the free ends of the two elastic bodies 1 is connected with a joining strap 4 that can be with or without elasticity. Each joining strap 4 can have a generally B-shaped adjusting ring (not shown) for adjusting the length of the joining strap 4, which is disposed on the joining strap 4. The two joining straps 4 are respectively connected with two fastening pieces 5, 6 which can be fastened on each other.

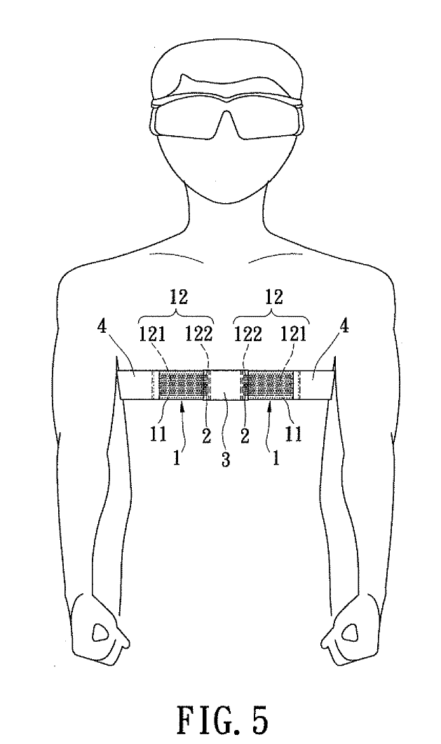

Accordingly, please refer to FIG. 5, during use, the elastic cloth device can surround a user's chest or head, or can also surround a user's arm or wrist etc, and can be positioned via the engagement of the fastening pieces 5, 6 in the embodiment. Based on the elastic tension of the elastic cloth straps 11 of the elastic bodies 1, the embodiment can cling to the user's body and the strain sensing units 12 can be properly attached to the user's skin, so that the elastic cloth straps 11 can drive the strain sensing units 12 to produce a physiological strain signal and the physiological strain signal is transmitted to the corresponding fastening elements 2 when the user's body produces a physiological strain reaction which has an effect on the elastic cloth straps 11 of the elastic bodies 1, thereby achieving the same object of detecting physiological signals such as electrocardiograms, brain waves, temperatures, pulses and so on.

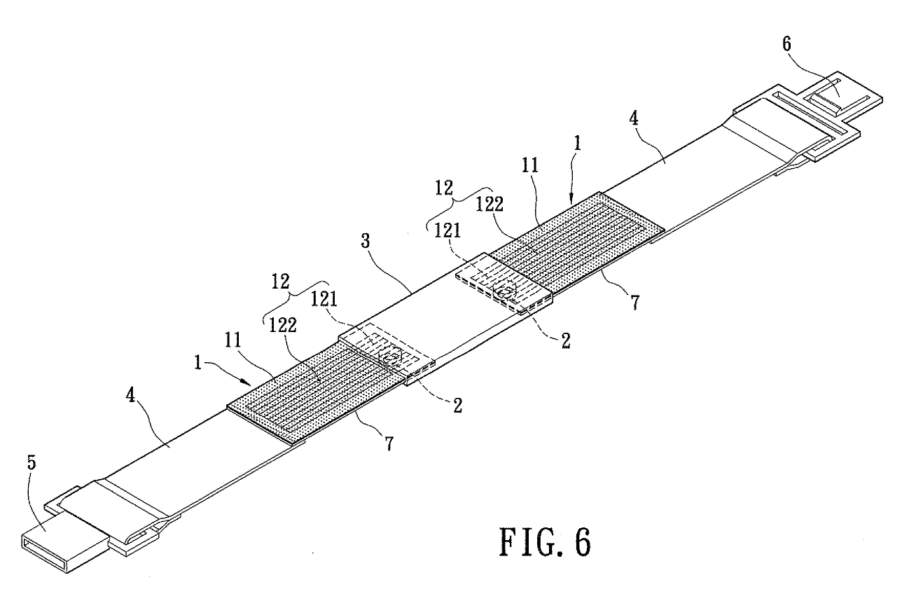

Additionally, please refer to FIG. 6, for keeping the humidity of the elastic cloth straps 11 so as to improve the electrical conductivity during signal detection, an alternative embodiment of the present invention further includes elastic water locking cloths 7 sewn on the other surfaces of the elastic cloth straps 11, corresponding to the strain sensing units 12, so as to provide a retractility effect that function in conjunction with the elastic effect of the elastic cloth straps 11, thereby the elastic cloth device of the present invention can be adapted to be used on various parts of the body due to its flexibility.

Consequently, the elastic cloth device with a function of physiological detection of the present invention can be worn on users' bodies, such as hands, palms, chests, or heads at all times to detect users' physiological signals generated by the users' physiological reaction, such as heart beats (which enables the generation of pulse information, temperature, electrical activity of the brain (which enables the generation of brain wave diagram), electrical activity of the heart over time (which enables the generation of physiological signals to form electrocardiograms)). Based on the elastic tension of the elastic bodies 1, the elastic cloth device of the present invention can be securely attached to users' bodies according to users' body types, thereby achieving a better usage effect and able to be adapted for more parts of the body intended for detection. Further, the strain sensing units 12 also have elasticity, so they can elastically cling to users' skins to detect the physiological strain reactions through the elastic cloth 11, so as to generate a physiological strain signal; and the strain sensing units 12 can also have large contact areas so that the physiological strain reaction can be detected with greater strength and accuracy.

What are disclosed above are only the specification and the drawings of the preferred embodiments of the present invention and it is therefore not intended that the present invention be limited to the particular embodiments disclosed. It will be understood by those skilled in the art that various equivalent changes may be made depending on the specification and the drawings of the present invention without departing from the scope of the present invention.

What is claimed is:

1. An elastic cloth device with a function of physiological detection, comprising:
an elastic body, including an elastic cloth strap and at least one strain sensing unit elastically woven with the elastic cloth strap, the strain sensing unit having a surface emerging from the elastic cloth strap; and
a fastening element with electrical conductivity, combined on the elastic body and electrically coupled with the strain sensing unit;
whereby when a physiological strain reaction has an effect on the elastic cloth strap, the elastic cloth strap drives the strain sensing unit to produce a physiological strain signal and the physiological strain signal is electrically transmitted to the fastening element.

2. The elastic cloth device as claimed in claim 1, wherein the strain sensing unit is a plurality of strain sensing conductive yarns.

3. The elastic cloth device as claimed in claim 1, wherein the strain sensing unit has a first portion and a second portion integrally and continuously connected with the first portion, the first portion emerges from a surface of the elastic cloth strap, and the second portion contacts the fastening element.

4. The elastic cloth device as claimed in claim 3, wherein the second portion is submerged in the elastic cloth strap, or submerged in the elastic cloth strap for a distance and then emerges from the surface of the elastic cloth strap, or the second portion directly extends to the fastening element from the surface of the elastic cloth strap.

5. The elastic cloth device as claimed in claim 1, wherein one strain sensing unit or more than one strain sensing units that is non-conductive to each other being disposed side by side are combined on the elastic cloth strap, wherein the one or more strain sensing units are long-strip-shaped.

6. The elastic cloth device as claimed in claim 1, wherein the fastening element is a male fastening element or a female fastening element, or a plurality of male fastening elements or female fastening elements, corresponding to a plurality of strain sensing units, wherein the strain sensing units are long-strip-shaped.

7. An elastic cloth device with a function of physiological detection, comprising:
two elastic bodies, adjacent to each other at intervals in turn and each including an elastic cloth strap and at least one strain sensing unit woven with the elastic cloth strap, the strain sensing unit having a surface emerging from the elastic cloth strap;
two fastening elements with electrical conductivity, each combined with one end of the corresponding elastic body and electrically coupled with the corresponding strain sensing unit; and
a connecting strap, connected with the ends of the elastic bodies so that the elastic bodies are spacedly connected in series;
whereby when a physiological strain reaction has an effect on the elastic cloth straps, the elastic cloth straps drive the strain sensing units to produce a physiological strain signal, and the physiological strain signal is electrically transmitted to the fastening elements.

8. The elastic cloth device as claimed in claim 7, wherein the strain sensing units are a plurality of strain sensing conductive yarns.

9. The elastic cloth device as claimed in claim 7, wherein each of the strain sensing units has a first portion and a second portion integrally and continuously connected with the first portion, the first portions emerge from surfaces of the elastic cloth straps, and the second portions contact the fastening elements.

10. The elastic cloth device as claimed in claim 9, wherein the second portions are submerged in the elastic cloth straps, or submerged in the elastic cloth straps for a distance and then emerge from the surfaces of the elastic cloth straps, or the second portions directly extend to the fastening elements from the surfaces of the elastic cloth straps.

11. The elastic cloth device as claimed in claim 7, wherein each of free ends of the elastic bodies is connected with a joining strap, and the joining straps are connected with fastening pieces which are fastened on each other.

12. The elastic cloth device as claimed in claim 11, wherein each joining strap has an adjusting ring for adjusting length of the joining strap disposed thereon.

13. The elastic cloth device as claimed in claim 7, wherein one strain sensing unit or more than one strain sensing units that is not conductive to each other being disposed side by side are combined on each elastic cloth strap, wherein the one or more strain sensing units are long-strip-shaped.

14. The elastic cloth device as claimed in claim 7, wherein each of the fastening elements is a male fastening element or a female fastening element, or a plurality of male fastening elements or female fastening elements, corresponding to a plurality of long-strip-shaped strain sensing units.

15. The elastic cloth device as claimed in claim 7, further comprising two elastic water locking cloths combined on the other surfaces of the elastic cloth straps, corresponding to the strain sensing units.
